(19) United States
(12) Patent Application Publication
(10) Pub. No.: US 2025/0258051 A1
(43) Pub. Date: Aug. 14, 2025

(54) APPARATUS FOR APPLYING TENSION TO LENGTHS OF FLEXIBLE MATERIAL

(71) Applicant: Oregon State University, Corvallis, OR (US)

(72) Inventors: Ravi Balasubramanian, Corvallis, OR (US); Benjamin Phelps, Eugene, OR (US); Hantao Ling, Milwaukie, OR (US); Justin C. Casebier, Portland, OR (US)

(73) Assignee: Oregon State University, Corvallis, OR (US)

(21) Appl. No.: 19/048,616

(22) Filed: Feb. 7, 2025

Related U.S. Application Data
(60) Provisional application No. 63/551,421, filed on Feb. 8, 2024.

(51) Int. Cl. G01L 5/06; A61F 2/08; G01L 5/10

(52) U.S. Cl. CPC G01L 5/06; A61F 2/08; G01L 5/10; A61F 2240/001

(57) ABSTRACT

An apparatus for applying tension to flexible lengths of material which can enable the user to pick up a flexible length of material, apply a specified tension, hold the material at that tension, and then mount the material on a surface without a change in the applied tension. The apparatus can include a boom arm and a main plate, where the main plate comprises a plunger assembly, a first support member, a second support member, and a force measurement device.

### CROSS REFERENCE TO RELATED APPLICATION

This application claims the benefit of U.S. Provisional Patent Application No. 63/551,421, filed Feb. 8, 2024, which is incorporated herein by reference in its entirety.

### ACKNOWLEDGEMENT OF GOVERNMENT SUPPORT

The invention was made with government support under Grant No. 2016530 awarded by the National Science Foundation. The government has certain rights in the invention.

### FIELD

The present application relates to an apparatus for applying tension to flexible lengths of material. This apparatus allows a user to place a flexible length of material, such as a tendon, into the apparatus and then apply a desired amount of tension to the material.

### BACKGROUND

It is often necessary to mount a flexible length of material on a surface. Flexible lengths of material can include wires, cables, ropes, or soft connective tissues such as tendons and ligaments. After applying a specific amount of tension to a flexible material it can often be difficult to hold the material at that tension while simultaneously mounting it on a surface. In many settings, even a minor departure from the desired tension can have detrimental effects.

For example, it is common in many medical settings, such as research, surgery, etc. for a flexible length of material, such as a tendon to have as specific tension applied to it before it is used. One specific example is in the research setting where it is often required to apply a specific amount of loading to a tendon to be studied, for example in an ex vivo bioreactor system. Furthermore, these applications often require high throughput with a low tolerance for deviations or errors between samples. Personnel working in the above settings need to be sure that the material they are working with is under the appropriate tension. Accordingly, a need exists for improved apparatus for applying tension to flexible lengths of material.

### SUMMARY

Described herein is an apparatus for applying tension to flexible lengths of material. This apparatus enables the user to pick up a flexible length of material, apply a specified tension, hold the material at that tension, and then mount the material on a surface while maintaining or substantially maintaining (e.g., depending on the material's elasticity) the applied tension. Example applications include biomedical engineering, robotic surgery, electronics assembly, etc.

In some examples, the apparatus can include a support; and a main plate coupled to the support; a plunger assembly, wherein the plunger assembly comprises a fixed portion which is coupled to the main plate and a travel element coupled to the fixed portion and movable in a first direction relative to the main plate; a force measurement device coupled to the main plate; a first support member coupled to the main plate and extending in the first direction from the main plate, wherein a distal end portion of the first support member comprises a first gripper; and a second support member coupled to the force measurement device and extending in the first direction, the second support member being on the opposite side of the plunger assembly from the first support member, wherein a distal end portion of the second support member comprises a second gripper, wherein the force measurement device is configured to measure force applied to the second support member in the first direction.

In some examples the force measurement device is a spring scale. In some examples, the force measurement device comprises a digital scale.

In some examples, the first gripper and second gripper are reverse action forceps. In some examples, the second gripper is lighter than the first gripper.

In some examples, the plunger assembly comprises a lead screw extending through the fixed portion and rotatably coupled to the fixed portion and the travel element is threadedly coupled to the lead screw. In some examples, rotation of the lead screw results in motion of the travel element axially in the first direction.

In some examples, a roller holder is coupled to a distal end portion of the travel element, wherein the roller holder comprises one or more rollers rotatably coupled to the roller holder. In some examples, the roller holder comprises a central portion and further comprises a first arm and a second arm coupled to the central portion and extending in the first direction from the central portion. In some examples, the one or more rollers comprises a first roller rotatably coupled to a distal end of the first arm and a second roller rotatably coupled to a distal end of the second arm.

In some examples, a method of using the apparatus includes engaging a first end portion of a flexible length of material with the first gripper, engaging a second end portion of the flexible length of material with the second gripper, and moving the travel element in the first direction to apply force to the flexible length of material between the first end portion and the second end portion.

In some examples, the method of using the apparatus further comprises measuring the force applied to the flexible length of material using the force measurement device. In some examples, the method of using the apparatus further comprises sterilizing the apparatus before engaging the first end portion of the flexible length of material with the first gripper.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of an apparatus for applying tension to a flexible length of material, according to one example.

FIG. 1B is a close-up perspective view of a first arm and a roller of the apparatus of FIG. 1A.

FIG. 2 is a perspective view of the main plate and the upper portion of the components coupled to the main plate of the apparatus of FIG. 1A.

FIG. 3 is a perspective view of the main plate and the lower portion of the components coupled to the main plate of the apparatus of FIG. 1A.

FIG. 4 is an exploded view of the components of the plunger assembly of the apparatus of FIG. 1A.

FIG. 5 is a cross sectional view of the top portion of the plunger assembly of the apparatus of FIG. 1A.

FIGS. 6A-6B show the apparatus of FIG. 1A as it picks up a piece of flexible material.

FIGS. 7A-7B show the apparatus of FIG. 1A as tension is applied to a piece of flexible material.

FIG. 8 is a perspective view of an apparatus for applying tension to a flexible length of material, according to another example.

### DETAILED DESCRIPTION

### General Considerations

The systems and methods described herein, and individual components thereof, should not be construed as being limited to the particular uses or systems described herein in any way. Instead, this disclosure is directed toward all novel and non-obvious features and aspects of the various disclosed embodiments, alone and in various combinations and subcombinations with one another. For example, any features or aspects of the disclosed embodiments can be used in various combinations and subcombinations with one another, as will be recognized by an ordinarily skilled artisan in the relevant field(s) in view of the information disclosed herein. In addition, the disclosed systems, methods, and components thereof are not limited to any specific aspect or feature or combinations thereof, nor do the disclosed things and methods require that any one or more specific advantages be present or problems be solved.

As used in this application the singular forms “a,” “an,” and “the” include the plural forms unless the context clearly dictates otherwise. Additionally, the term “includes” means “comprises.” Further, the term “coupled” encompasses mechanical, electrical, magnetic, optical, as well as other practical ways of coupling or linking items together, and does not exclude the presence of intermediate elements between the coupled items. Furthermore, as used herein, the term “and/or” means any one item or combination of items in the phrase.

As used herein, the term “exemplary” means serving as a non-limiting example, instance, or illustration. As used herein, the terms “e.g.,” and “for example,” introduce a list of one or more non-limiting embodiments, examples, instances, and/or illustrations.

As used herein, directional words such as “above,” “under,” “downward,” “upward,” and “beneath” are also to be understood as they would be with reference to a user of the apparatus, i.e., “beneath” is closer to the ground “above” is farther from the ground as seen from a frame of reference when the apparatus for applying tension to flexible lengths of material is in use, for example on a lab table.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed things and methods can be used in conjunction with other things and methods. Additionally, the description sometimes uses terms like “provide,” “produce,” “determine,” and “select” to describe the disclosed methods. These terms are high-level descriptions of the actual operations that are performed. The actual operations that correspond to these terms will vary depending on the particular implementation and are readily discernible by one of ordinary skill in the art having the benefit of this disclosure.

### Exemplary Apparatus for Tensioning a Flexible Material

The present disclosure provides an apparatus for picking up a flexible length of material, applying a specified tension, holding it at that tension, and then mounting it on a surface. In some instances, the apparatus can include a base, a boom arm, and a main plate, where the main plate provides support for several other components. These components can include a plunger assembly, a first support member, a second support member, and a force measurement device. As described below in more detail, the operator can position the flexible length of material by gripping it in grippers positioned at end portions of the first and second support members, apply tension by adjusting the position of the plunger assembly, and measure the force applied with the force measurement device.

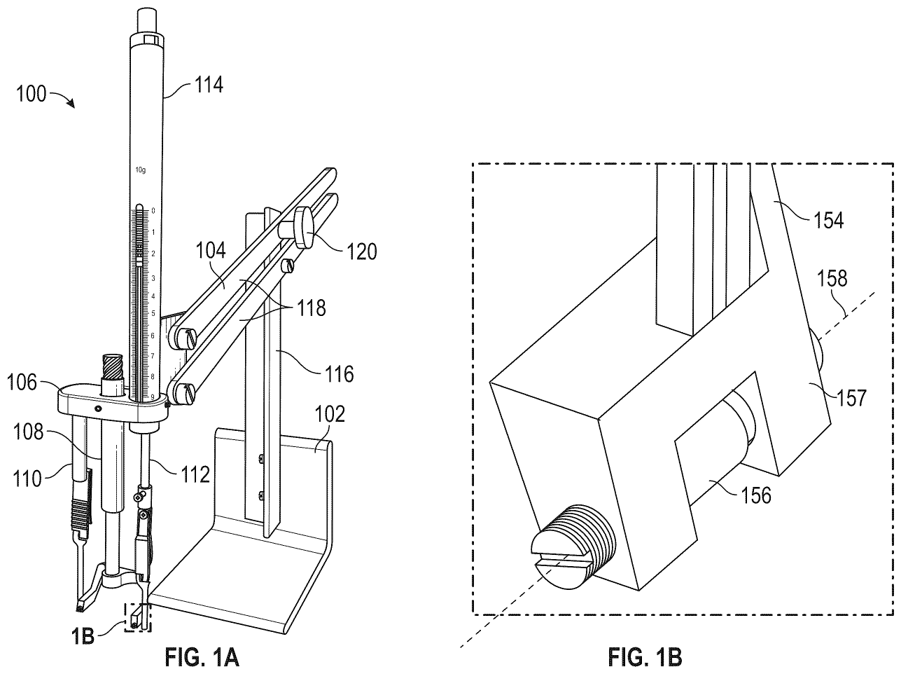

FIG. 1A depicts an exemplary apparatus 100 for applying tension to a flexible length of material including examples of a base 102, a support configured as a boom arm 104, and a holder configured as a main plate 106. The main plate 106 is connected to a plunger assembly 108, a first support member 110, a second support member 112 (via the force measuring device), and a force measurement device 114.

The base 102 serves as a support for the rest of the apparatus and provides stability to the apparatus 100. The base 102 may be connected to a vertical support, such as vertical post 116, and the boom arm 104 may be coupled to the top of the vertical support 116. In the depicted example, the base 102 is constructed of two rectangular pieces, one in a horizontal plane and another in a vertical plane. In some examples, the base 102 can be shaped in any of a variety of shapes such as a circle, square, etc. In some examples, the base 102 may be of a three-dimensional shape such as a cubic, cylindrical, or spherical, etc. In some examples, the boom arm 104 can be supported by other means, such as a clamp which is configured to clamp onto another structure such as a lab bench or a surgical table. In other examples, the boom arm 104 may be fixed directly to a structure such as a wall or other vertical surface by a permanent or semi-permanent connection, such as by bolts, screws, pins or by other similar means.

In the depicted example, the boom arm 104 comprises two parallel members 118 and a position lock knob 120. The members 118 can be fixed at a first end portion to the vertical support 116 and at a second end portion to the main plate 106 by a permanent or semi-permanent connection, such as by bolts, screws, pins or by other means, in a way that allows each member to rotate about a single point on the first end portion and a single point on the second end portion. The position lock knob 120 can be tightened to prevent the two parallel members 118 from moving once they are in the desired position. In other examples the boom arm 104 may be of another type that allows the position of the apparatus to be adjusted, such as a scissor linkage, an arm with one or more elbow joints, or another similar structure.

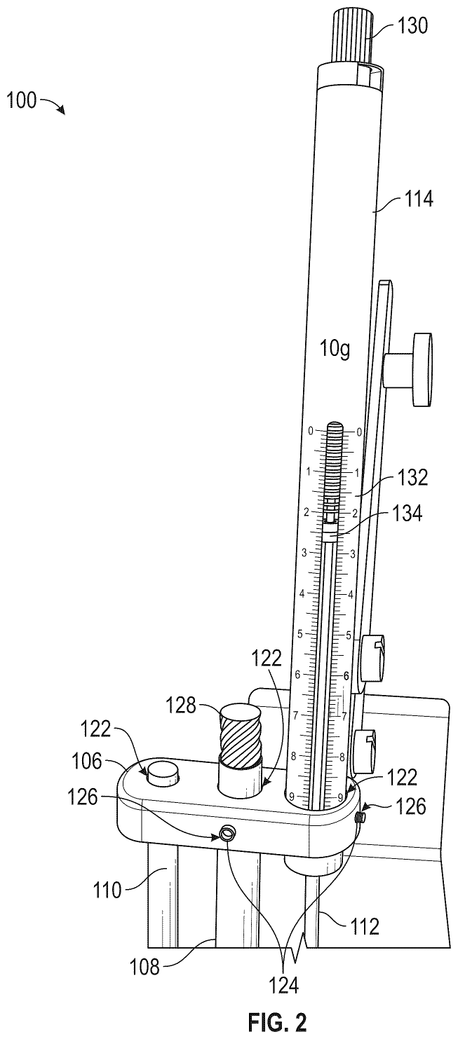

As depicted in FIG. 1A, the main plate 106 is coupled to the boom arm 104 and provides a platform to which several other components are coupled. As depicted in FIG. 2, the main plate 106 is coupled to a plunger assembly 108, a first support member 110, and a force measurement device 114. The second support arm 112 is mounted to the bottom of the force measurement device 114, or can be part of the force measurement device 114. In the depicted example, the main plate 106 is rectangular with rounded corners, however, in other examples, it could be square, oval, or any other suitable shape. In some examples the main plate 106 is constructed from a bar of 303 stainless steel. In some examples, another material such as metal, plastic, composite, or any suitable material may be used.

As depicted, in some examples, the main plate 106 comprises a unitary piece. In some examples, the main plate can comprise multiple pieces such that the length of the main plate 106 can be adjustable so that the distance between the first support member 110 and the second support member 112 can be changed to allow the apparatus to pick up flexible materials of varying lengths.

In some examples the main plate comprises several apertures 122 for mounting the other components such as the plunger assembly 108, the first support member 110, and the force measurement device 114. In the depicted example the apertures 122 are round, but can be shaped to match the component to be received. In some examples, the components are held in place by set screws 124. These set screws are inserted into threaded holes 126 that are perpendicular to the apertures 122. When inserted and tightened the set screws 124 apply a force which holds the plunger assembly 108, a first support member 110, and a force measurement device 114 in place on the main plate 106. In some examples, the components may be held in place by other means such as a pressed fit, autoclave-rated adhesive, or another permanent or semi-permanent connection, such as by bolts, screws, pins or by other similar means.

FIG. 2 depicts examples of components that extend from the main plate. These components include a knob 128 which is used to operate the plunger assembly 108 and an upper portion of the force measurement device 114.

The force measurement device 114 measures the force applied to the second support member 112. The upper portion of the force measurement device 114 is depicted in FIG. 2 and may include features which allow the operation and adjustment of the force measurement device 114. In the depicted example, the force measurement device is a mechanical spring scale. The upper portion contains a scale knob 130, a graduated marking 132, and a pointer 134. The scale knob 130 can be used to tare and/or calibrate the scale. The graduated markings 132 are comprised of divisions of equal length marked on the outer surface of the scale which constitute the units of measurement. The pointer 134 is connected to the internal spring and moves along the graduated markings 132 to indicate the amount of force applied. In some examples, the force measurement device 14 can be another suitable scale such as a dial scale.

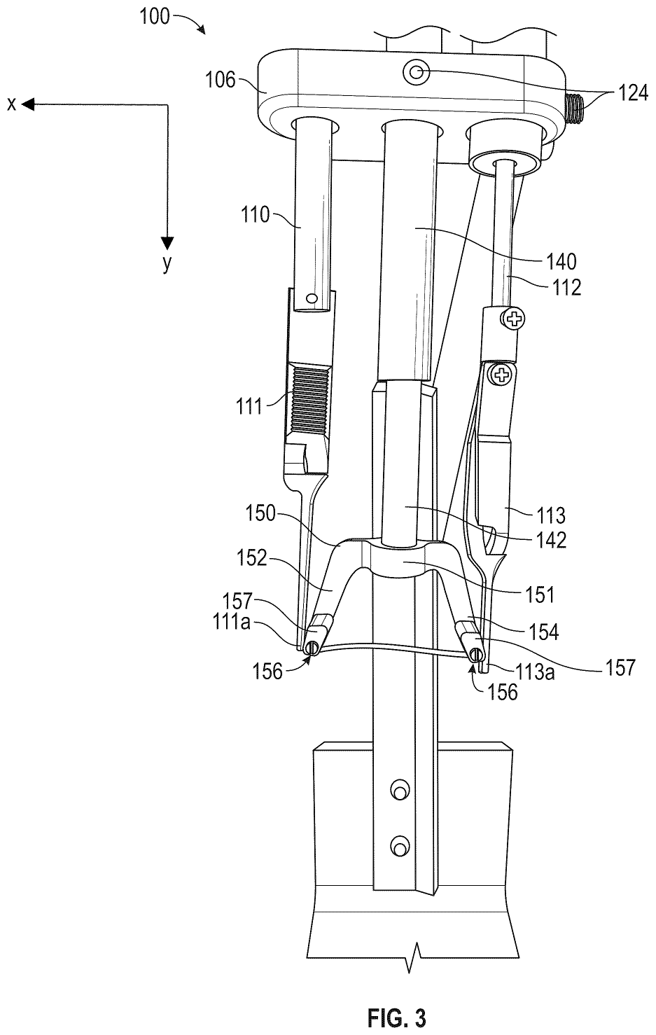

FIG. 3 depicts several components which extend downward (e.g., in a first direction) from the main plate 106, in the depicted example the components extend along the y-axis. The upper portions of the first support member 110 and the plunger assembly 108 are coupled to the main plate 106 as described above. The upper portion of the second support member 112 is coupled to the bottom portion of the force measurement device 114, which is in turn coupled to the main plate 106 as described above. The first support member 110 and the second support member 112 may be coupled to a first gripper 111 and a second gripper 113, respectively, at their bottom end portions. The first gripper 111 has a first gripping portion 111a (e.g., a pair of jaws or clamps) and the second gripper has a second gripping portion 113a (e.g., a pair of jaws or clamps). The first gripper 111 and second gripper 113 may be reverse action forceps as depicted. In other examples, the first gripper 111 and the second gripper 113 may also be any other suitable mechanism such as Kelly forceps or any other type of clamp capable of gripping and holding a flexible length of material. By coupling the second support member 112 to the bottom portion of the force measurement device 114 (e.g., to the internal member that moves longitudinally when the spring is compressed and released) downward force applied to the second support member 112 can be measured by the force measurement device 114. In the depicted example, the second support member 112, including the second gripper 113, is sized and shaped to be smaller and lighter than the first support member 110 and first gripper 111. This has the benefit of reducing the weight of the second support member 112 and the associated effect on the force measurement device 114.

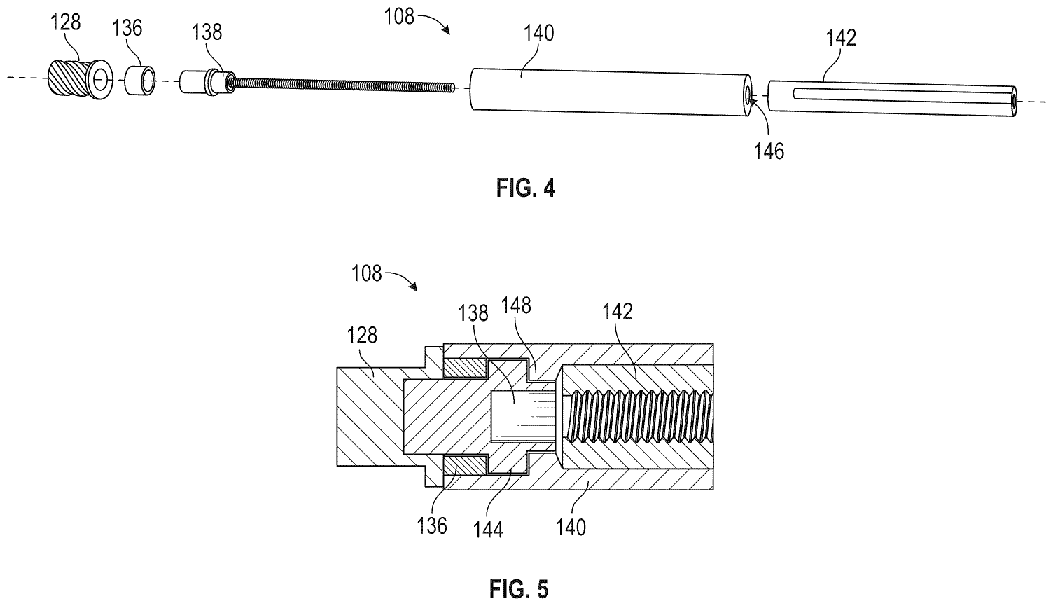

FIGS. 4-5 depict components of an example of the plunger assembly (also referred to as a “linear actuator assembly”) 108. FIG. 4 depicts an exploded view of several components of the plunger assembly 108 including the knob 128, a ring 136, a lead screw element 138, a housing cylinder 140, and a travel element 142. The upper portion of the lead screw element 138 can include a portion 144 which has a larger radius than the rest of the lead screw element 138. The housing cylinder 140 includes a bore 146 in which the ring 136, the lead screw element 138, and the travel element 142 can be positioned. As depicted in FIG. 5, the portion 144 of the lead screw element 138 can sit on top of an internal flange 148 in the bore 146 of the housing cylinder 140. The ring 136 is sized to pressure fit into the bore 146 of the housing cylinder 140 but have a free fit around the lead screw element 138. In some examples, the ring 136 may also be coupled to the bore using other means such as, for example, an autoclave-rated glue. In this way, the lead screw element 138 is prevented from moving up or down by the portion 144 positioned between the internal flange 148 and the ring 136, but the lead screw element 138 is free to rotate inside the main cylinder.

The lead screw element 138 is threadedly coupled with the travel element 142 and together may be referred to as a “drive”, which is also sized to fit within the bore 146 of the housing cylinder 140. As the lead screw element 138 is rotated, the travel element 142 moves in the axial direction. In some instances, a different type of plunger assembly may be used to provide the downward axial force such as, for example, a hydraulic cylinder, a pneumatic actuator, an electromechanical actuator such as a voice coil motor, or another similar device.

As depicted in FIG. 3, the travel element 142 can be coupled at its bottom end portion to a plunger head configured as a roller holder 150. In the depicted example, the roller holder 150 comprises a central portion 151, first arm 152, and a second arm 154. In some examples, each of the arms 152, 154 extend from the central portion 151 in the first direction (in the depicted example, along the y-axis). In some examples, the central portion and the arms 152, 154 are integrally formed. In some examples the arms 152, 154 are coupled to the central portion 151 by a permanent or semi-permanent connection, such as by bolts, screws, pins or by other similar means. Each arm 152, 154 can comprise (e.g., house) one or more rollers 156. In the illustrated example the rollers 156 can be disposed in a corresponding roller mount (also referred to as a “roller housing”) 157 at an end portion of the respective arm, and can be rotatable around a roller axis 158 (FIG. 1B) that can extend out of the page in FIG. 3. In some examples the rollers are supported by bearings, such as, for example, jewel bearings. In the depicted example, there are two rollers 156, one roller 156 is rotatably coupled to the end portion of the first arm 152 and a second roller 156 is rotatably coupled to the end portion of the second arm. In some examples the roller holder 150 is adjustable so that the distance between the first arm 152 and second arm 154 can be changed to allow the apparatus to apply tension to flexible materials of varying lengths.

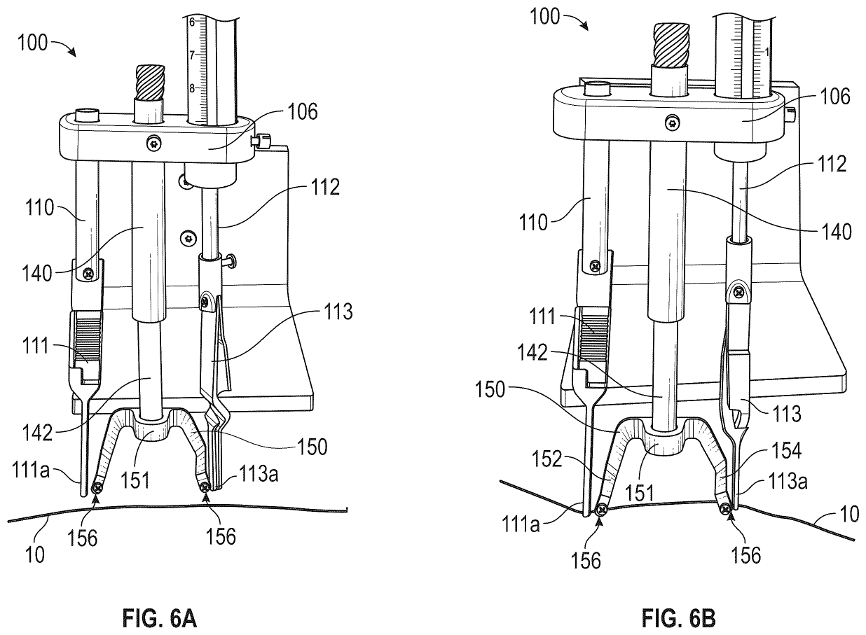

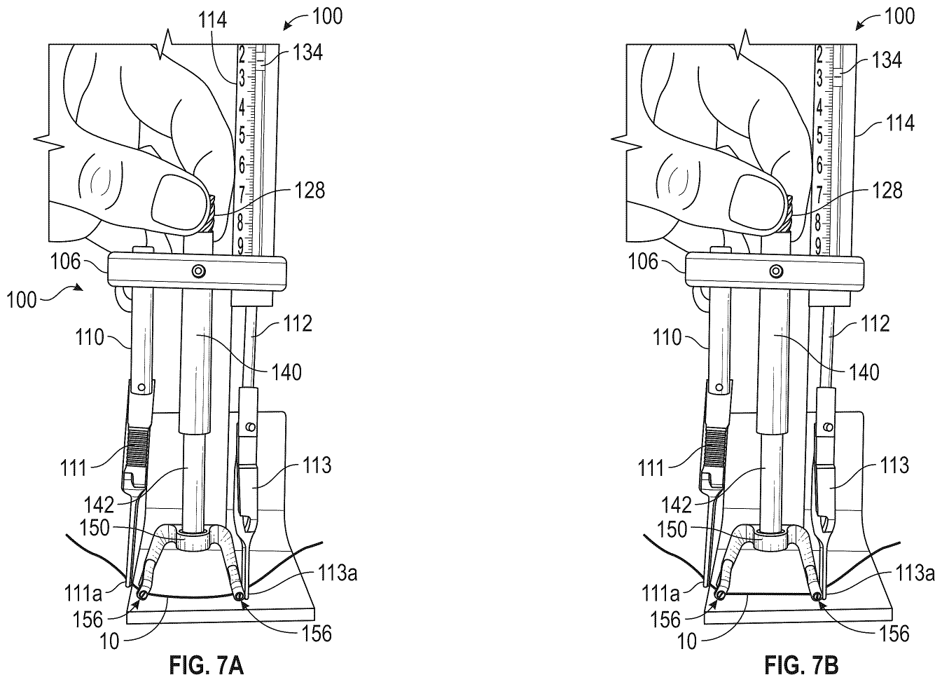

In use, as depicted in FIGS. 6A-6B and 7A-7B, a length of flexible material such as a tendon 10 can be gripped by the apparatus and tensioned to a specified tension. The process of positioning the tendon 10 in the apparatus 100 is depicted in FIGS. 6A-6B. In FIG. 6A the tendon 10 is not inserted into the apparatus 100 and has no tension applied to it, i.e., it is in an initial relaxed state beneath the apparatus. In FIG. 6B, the griping portion 111a of the first gripper 111 grips the tendon 10 at a first location along its length and the second gripping portion 113a of the second gripper 113 grips the tendon at a second location along its length. The tendon 10 is positioned under the rollers 156 of the first arm 152 and the second arm 154, but no tension is applied yet.

FIGS. 7A-7B depict tension being applied to the tendon 10. In FIG. 7A, the tendon 10 has been picked up off the surface and is held in (e.g., engaged by) the first gripping portion 111a of the first gripper 111 and the second gripping portion 113a the second gripper 113. In FIG. 7A, no downward pressure has yet been applied to the tendon 10. In FIG. 7B the knob 128 has been adjusted, causing the lead screw element 138 to rotate as described above, and the travel element 142 threadedly coupled with the lead screw element 138 to move downward. This moves the roller holder 150 downwardly, changing the position of the roller holder relative to the ends of the grippers such that the rollers 156 push on the tendon 10 and apply tension to the part of the tendon 10 held between the first gripper 111 and the second gripper 113. The force applied to the tendon 10 by the roller holder 150 is transmitted to the measurement device 114 by the second support member 112. The downward force is measured by the force measurement device 114, and is indicated by the pointer 134, which is seen to move downward along the graduated markings 132 in FIG. 7B relative to FIG. 7A.

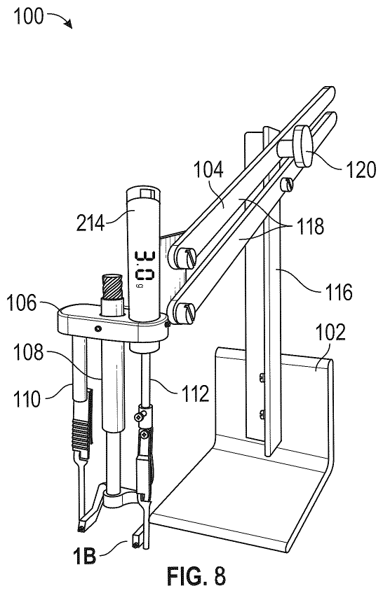

In certain examples the tensioner apparatus 100 can include a force measurement device that is a digital scale or other electronic force measurement device. For example, FIG. 8 illustrates another example of the apparatus 100 in which the force measurement device is a digital scale 214. A digital scale can have several advantages, for example increased accuracy, sensitivity, etc.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The apparatus described herein can be used to tension any of a variety of relatively long, flexible materials including natural materials such as tendons and ligaments, but also string, yarn, thread, cord, twine, rope, fabric, etc. Additionally, although the force measurement apparatus is depicted as part of the second support member on the right side of the device, in other examples the force measurement device can be on the left side and/or can be part of the first support member. The main plate can also be coupled to any of the various supports that can position the main plate over a workpiece, and such supports can be configured differently than the boom arm 104.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An apparatus for tensioning a flexible length of material, the apparatus comprising: a base; a boom arm coupled to the base; and a main plate coupled to the boom arm, the main plate comprising: a plunger assembly, wherein the plunger assembly extends in a first direction from the main plate and comprises: a fixed portion coupled to the main plate; a lead screw extending through the fixed portion and rotatably coupled to the fixed portion; a travel element threadedly coupled to the lead screw, and a roller holder coupled to a distal end of the travel element and comprising one or more rollers rotatably coupled to the roller holder; a force measurement device coupled to the main plate; a first support member coupled to the main plate and extending in the first direction from the main plate, wherein a distal end portion of the first support member comprises a first gripper; and a second support member coupled to the force measurement device and extending in the first direction, the second support member being on the opposite side of the plunger assembly from the first support member, wherein a distal end portion of the second support member comprises a second gripper, wherein rotation of the lead screw moves the travel element and the roller holder axially in the first direction, and wherein the force measurement device is configured to measure force applied to the second support member in the first direction.

Example 2. The apparatus of any example herein, particularly example 1, wherein the force measurement device is a spring scale.

Example 3. The apparatus of any example herein, particularly example 1, wherein the force measurement device is a digital scale.

Example 4. The apparatus of any example herein, particularly any one of examples 1-3, wherein the first gripper and the second gripper are reverse action forceps.

Example 5. The apparatus of any example herein, particularly any one of any one of examples 1-4, wherein the roller holder comprises a central portion and further comprises a first arm and a second arm coupled to the central portion and extending in the first direction from the central portion.

Example 6. The apparatus of any example herein, particularly example 5, wherein the one or more rollers comprises two rollers, a first roller rotatably coupled to a distal end of the first arm and a second roller rotatably coupled a distal end of the second arm.

Example 7. An apparatus for tensioning a flexible length of material, the apparatus comprising: a support; and a main plate coupled to the support; a plunger assembly, wherein the plunger assembly comprises a fixed portion which is coupled to the main plate and a travel element coupled to the fixed portion and movable in a first direction relative to the main plate; a force measurement device coupled to the main plate; a first support member coupled to the main plate and extending in the first direction from the main plate, wherein a distal end portion of the first support member comprises a first gripper; and a second support member coupled to the force measurement device and extending in the first direction, the second support member being on the opposite side of the plunger assembly from the first support member, wherein a distal end portion of the second support member comprises a second gripper, wherein the force measurement device is configured to measure force applied to the second support member in the first direction.

Example 8. The apparatus of any example herein, particularly example 7, wherein the force measurement device is a spring scale.

Example 9. The apparatus of any example herein, particularly example 7, wherein the force measurement device comprises a digital scale.

Example 10. The apparatus of any of any example herein, particularly any one of examples 7-9, wherein the first gripper and second gripper are reverse action forceps.

Example 11. The apparatus of any example herein, particularly any one of examples 7-10, wherein the second gripper is lighter than the first gripper.

Example 12. The apparatus of any example herein, particularly any one of examples 7-11, wherein the plunger assembly comprises a lead screw extending through the fixed portion and rotatably coupled to the fixed portion and the travel element is threadedly coupled to the lead screw.

Example 13. The apparatus of any example herein, particularly example 12, wherein rotation of the lead screw results in motion of the travel element axially in the first direction.

Example 14. The apparatus of any example herein, particularly any one of examples 7-13, further comprising a roller holder coupled to a distal end portion of the travel element, wherein the roller holder comprises one or more rollers rotatably coupled to the roller holder.

Example 15. The apparatus of any example herein, particularly example 14, wherein the roller holder comprises a central portion and further comprises a first arm and a second arm coupled to the central portion and extending in the first direction from the central portion.

Example 16. The apparatus of any example herein, particularly example 15, wherein the one or more rollers comprises a first roller rotatably coupled to a distal end of the first arm and a second roller rotatably coupled to a distal end of the second arm.

Example 17. A method of using the apparatus of any example herein, particularly the apparatus of example 7, the method comprising: engaging a first end portion of a flexible length of material with the first gripper, engaging a second end portion of the flexible length of material with the second gripper, and moving the travel element in the first direction to apply force to the flexible length of material between the first end portion and the second end portion.

Example 18. The method of any example herein, particularly example 17, further comprising measuring the force applied to the flexible length of material using the force measurement device.

Example 19. The method of any example herein, particularly any one of examples 17 or 18, further comprising sterilizing the apparatus before engaging the first end portion of the flexible length of material with the first gripper.

The features described herein with regard to any example can be combined with other features described in any one or more of the other examples, unless otherwise stated. For example, any one or more of the features of one exemplary apparatus can be combined with any one or more features of another exemplary apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is at least as broad as the following claims and equivalents of the recited features.

1. An apparatus for tensioning a flexible length of material, the apparatus comprising:
a base;
a boom arm coupled to the base; and
a main plate coupled to the boom arm, the main plate comprising:
a plunger assembly, wherein the plunger assembly extends in a first direction from the main plate and comprises:
a fixed portion coupled to the main plate;
a lead screw extending through the fixed portion and rotatably coupled to the fixed portion;
a travel element threadedly coupled to the lead screw, and
a roller holder coupled to a distal end of the travel element and comprising one or more rollers rotatably coupled to the roller holder;
a force measurement device coupled to the main plate;
a first support member coupled to the main plate and extending in the first direction from the main plate, wherein a distal end portion of the first support member comprises a first gripper; and
a second support member coupled to the force measurement device and extending in the first direction, the second support member being on the opposite side of the plunger assembly from the first support member, wherein a distal end portion of the second support member comprises a second gripper,
wherein rotation of the lead screw moves the travel element and the roller holder axially in the first direction, and
wherein the force measurement device is configured to measure force applied to the second support member in the first direction.

2. The apparatus of claim 1, wherein the force measurement device is a spring scale.

3. The apparatus of claim 1, wherein the force measurement device is a digital scale.

4. The apparatus of claim 1, wherein the first gripper and the second gripper are reverse action forceps.

5. The apparatus of claim 1, wherein the roller holder comprises a central portion and further comprises a first arm and a second arm coupled to the central portion and extending in the first direction from the central portion.

6. The apparatus of claim 5, wherein the one or more rollers comprises two rollers, a first roller rotatably coupled to a distal end of the first arm and a second roller rotatably coupled to a distal end of the second arm.

7. An apparatus for tensioning a flexible length of material, the apparatus comprising:
a support; and
a main plate coupled to the support;
a plunger assembly, wherein the plunger assembly comprises a fixed portion which is coupled to the main plate and a travel element coupled to the fixed portion and movable in a first direction relative to the main plate;
a force measurement device coupled to the main plate;
a first support member coupled to the main plate and extending in the first direction from the main plate, wherein a distal end portion of the first support member comprises a first gripper; and
a second support member coupled to the force measurement device and extending in the first direction, the second support member being on the opposite side of the plunger assembly from the first support member, wherein a distal end portion of the second support member comprises a second gripper,
wherein the force measurement device is configured to measure force applied to the second support member in the first direction.

8. The apparatus of claim 7, wherein the force measurement device is a spring scale.

9. The apparatus of claim 7, wherein the force measurement device comprises a digital scale.

10. The apparatus of claim 7, wherein the first gripper and second gripper are reverse action forceps.

11. The apparatus of claim 7, wherein the second gripper is lighter than the first gripper.

12. The apparatus of claim 7, wherein the plunger assembly comprises a lead screw extending through the fixed portion and rotatably coupled to the fixed portion and the travel element is threadedly coupled to the lead screw.

13. The apparatus of claim 12, wherein rotation of the lead screw results in motion of the travel element axially in the first direction.

14. The apparatus of claim 7, further comprising a roller holder coupled to a distal end portion of the travel element, wherein the roller holder comprises one or more rollers rotatably coupled to the roller holder.

15. The apparatus of claim 14, wherein each of the one or more rollers is supported by bearings.

16. The apparatus of claim 14, wherein the roller holder comprises a central portion and further comprises a first arm and a second arm coupled to the central portion and extending in the first direction from the central portion.

17. The apparatus of claim 16, wherein the one or more rollers comprises a first roller rotatably coupled to a distal end of the first arm and a second roller rotatably coupled to a distal end of the second arm.

18. A method of using the apparatus of claim 7, the method comprising:
engaging a first end portion of a flexible length of material with the first gripper,
engaging a second end portion of the flexible length of material with the second gripper, and
moving the travel element in the first direction to apply force to the flexible length of material between the first end portion and the second end portion.

19. The method of claim 18, further comprising measuring the force applied to the flexible length of material using the force measurement device.

20. The method of claim 18, further comprising sterilizing the apparatus before engaging the first end portion of the flexible length of material with the first gripper.
